(19) United States
(12) Plant Patent Application Publication
(10) Pub. No.: US 2013/0047306 P1
(43) Pub. Date: Feb. 21, 2013

(54) Raspberry plant named 'PACIFIC MAJESTY'

(50) ;

(71) Applicants: Pilar Bañados, Huechuraba (CL); A. Ellen Thompson, Fayetteville, AR (US); Miguel Alliende, Huechuraba (CL); Victor Moller, Huechuraba (CL); John R. Clark, Fayetteville, AR (US)

(72) Inventors: Pilar Bañados, Huechuraba (CL); A. Ellen Thompson, Fayetteville, AR (US); Miguel Alliende, Huechuraba (CL); Victor Moller, Huechuraba (CL); John R. Clark, Fayetteville, AR (US)

(73) Assignee: PACIFIC BERRY BREEDING, LLC, Salinas, CA (US)

(21) Appl. No.: 13/136,977

(22) Filed: Aug. 15, 2011

(51) Int. Cl. A01H 5/00

(52) U.S. Cl. PLT/204

(57) ABSTRACT

A new and distinct cultivar of Raspberry plant named ‘Pacific Majesty’ as described and shown herein. ‘Pacific Majesty’ provides typically larger fruit, better firmness and typically higher yields than ‘Rafzaqu’, U.S. Plant Pat. No. 19,512 and ‘Marcela’ U.S. Plant Pat. No. 17,819. ‘Pacific Majesty’ provides fruit with a lighter red color than ‘Pacific Royale’ U.S. Plant Pat. No. 21,536. Distinct characteristics of ‘Pacific Majesty’ include the combination of very large, conic fruit, bright color and high yield.

### BACKGROUND AND SUMMARY

Raspberries are a well-known, aggregate fruit grown commercially throughout the world. One example of an existing, patented raspberry variety is ‘Rafzaqu’ (marketed as ‘Himbo-Top’®), U.S. Plant Pat. No. 19,512. Another example of an existing, patented raspberry variety is ‘Pacific Royale’, U.S. Plant Pat. No. 21,536. A third example of an existing, patented variety is ‘Marcela’, U.S. Plant Pat. No. 17,819. Such varieties, however, do not provide large enough fruit, yield enough, or produce berries with bright enough color to be competitive in the commercial market. Thus, there is a need for raspberry varieties that provide larger fruit, brighter color, higher yields and that are competitive.

The present cultivar, ‘Pacific Majesty’, provides one or more of these, or other characteristics. For example, the present cultivar, ‘Pacific Majesty’, provides typically larger fruit than ‘Rafzaqu’ and ‘Marcela’. Further, ‘Pacific Majesty’ produces berries that are larger and of lighter red color as compared to ‘Pacific Royale’. ‘Pacific Majesty’ also produces higher yields than ‘Rafzaqu’, ‘Pacific Royale’, and ‘Marcela’. The fruits of ‘Pacific Majesty’ are also firmer than those of ‘Rafzaqu’. It should be noted that ‘Pacific Royale’ is a previous release from the same breeding program as the present cultivar, ‘Pacific Majesty’, and that ‘Pacific Majesty’ is most definitely an improvement over ‘Pacific Royale’, particularly in regard to fruit color. Distinct characteristics of ‘Pacific Majesty’ include the combination of very large fruit, light red color that darkens slightly in cold storage, firmness, long shelf life and high yield discussed herein.

### BRIEF DESCRIPTION OF THE PHOTOGRAPHS

FIG. 1 is a photograph of the whole plant of the Raspberry cultivar ‘Pacific Majesty’ produced from root cuttings, showing floricanes aged 12 months.

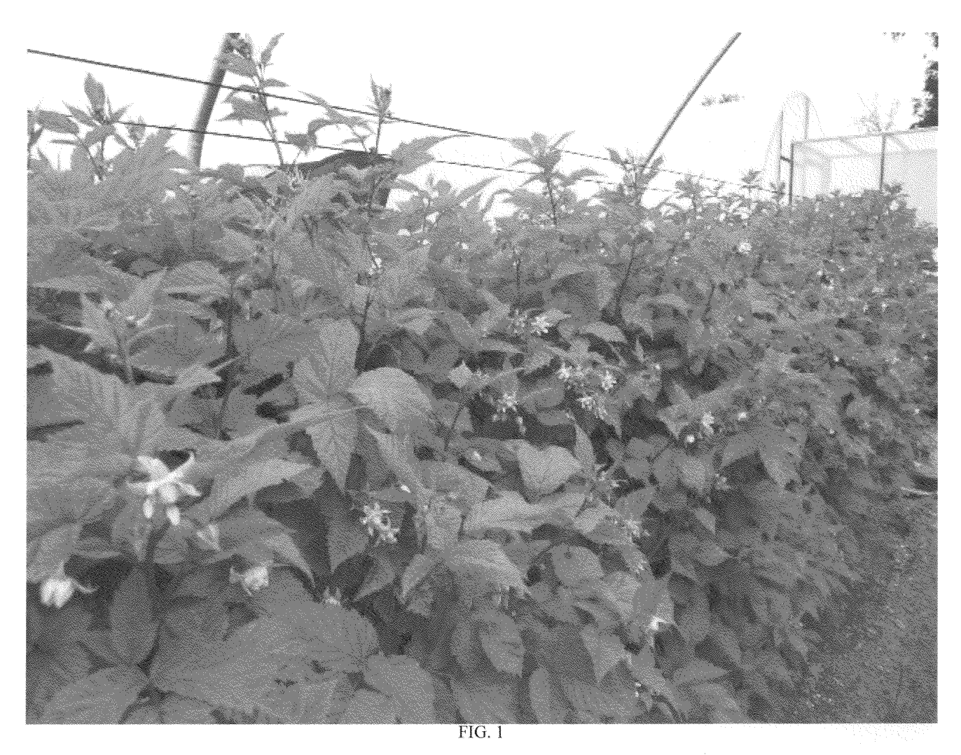

FIG. 2 is a photograph of primocane color and ripe fruit on primocanes of the Raspberry cultivar ‘Pacific Majesty’ produced from root cuttings, on primocanes aged 7 months.

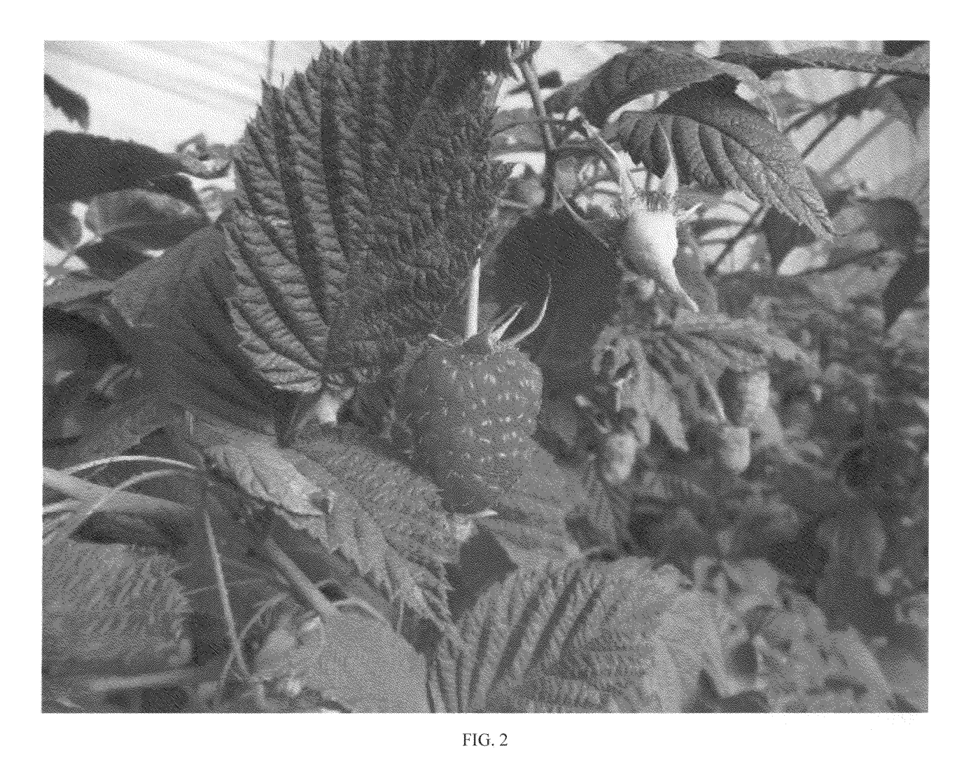

FIG. 3 is a close-up photograph of ripe and unripe fruit and leaf detail on floricanes of the Raspberry cultivar ‘Pacific Majesty’ produced from root cuttings, on primocanes aged 14 months.

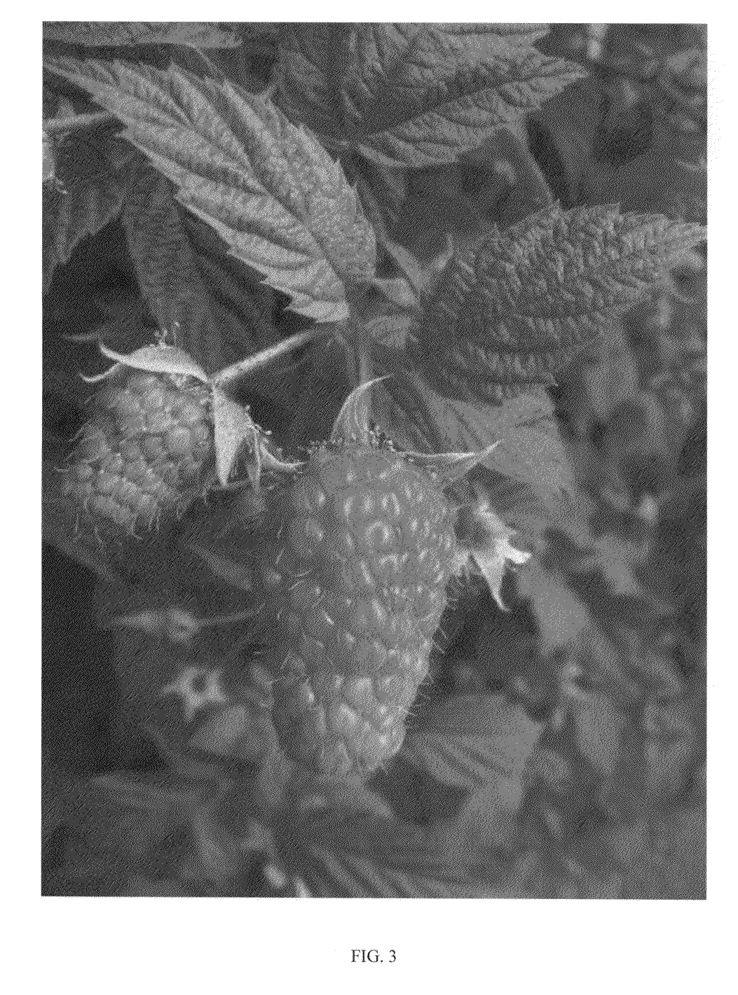

FIG. 4 is a photograph of the distinct leaf shape of the Raspberry cultivar ‘Pacific Majesty’ produced in vitro, aged 3.5 months.

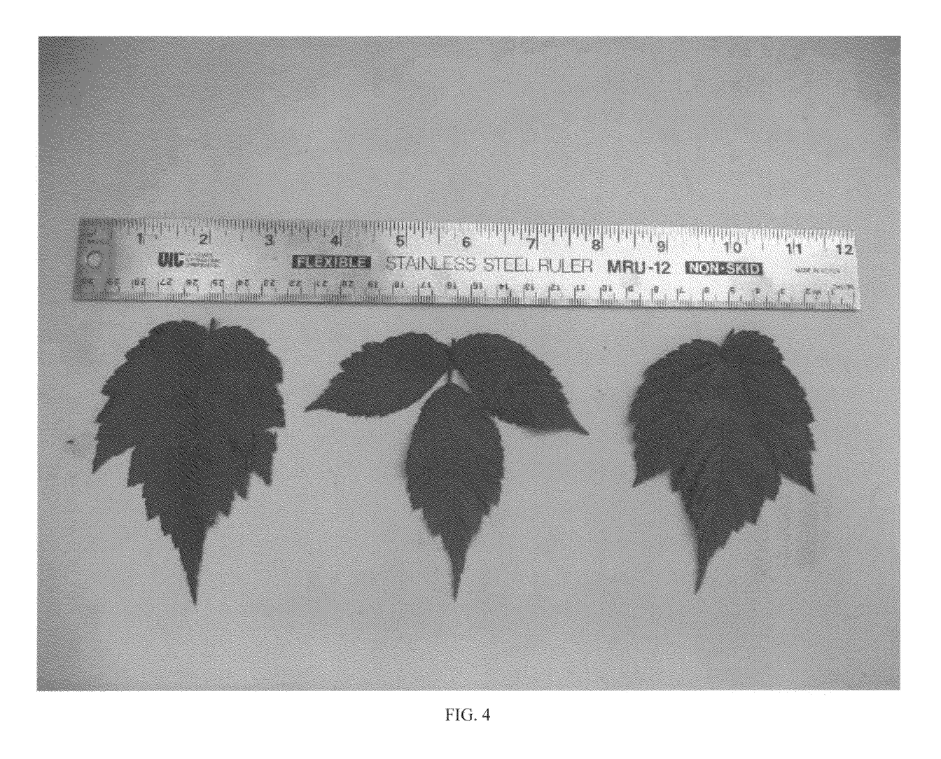

### DETAILED DESCRIPTION

Note: statements of characteristics herein represent exemplary observations of the cultivar herein and will vary depending on time of year, growing location, horticultural practices employed, annual weather, etc.
Cultivar name: ‘Pacific Majesty’
Classification:
Family: Rosaceae
Botanical name: Rubus idaeus
Common name: Raspberry
Parentage:

Female parent (please provide name or code): Unknown
Male parent (please provide name or code flower): Unknown
Growing location for the observations herein: Watsonville, Calif., USA. The variety was also tested in Zapotiltic, Jalisco, Mexico and was found to be adapted to that climate.
Time of year (season): Mid summer to Autumn
Age of plants used for this discussion: 7-9 months for primocanes; 12 months for floricanes
Age of plants used for the photographs in the figures: 7 months, primocanes from root cuttings and 3.5 months, primocanes from in vitro propagation.
Type of greenhouse covering or growing structure, or field: High tunnel over field
Light: Natural References to color refer to the R.H.S. — Fifth Edition
Plant:
Form/shape: Vase
Growth habit: Semi-Erect to Erect
Height: 150 cm to 200 cm, as measured from crown to apex (primocanes)
Spread: 34 cm, on average, as measured from distal leaf tip to distal leaf tip
How propagated: Root cuttings, in vitro. As of the filing of the current application, at least 10 generations of Pacific Majesty (primarily in vitro) have been created and the characteristics herein have been stable.
Time to initiate and develop roots: 25 days from etiolated root cuttings
Root description: Fibrous
Primocanes: (Note: Primocane data recorded on Oct. 21, 2010)
Diameter: 0.7 cm for a 1.6 m cane
Height: 140 to 160 cm
Number of nodes: 41 on average, for a 1.6 m cane
Internode length: 3.7 cm on average
Number of canes/hill: 10-12
Cane color: RHS 145A
Spine color: RHS 59A
Number of spines per unit area, the spine shape, spine length, spine width and spine apex descriptor:
   Spines/unit area.—3/cm².
   Spine shape.—Acuminate.
   Spine length.—0.1 cm.
   Spine width.—0.01 cm.
   Spine apex descriptor.—Acuminate.
Floricanes: (Note: Floricane data recorded on Apr. 2, 2011)
Diameter: 0.7 cm
Length: 100 to 115 cm
Number of nodes: 22 on average
Internode length: 4.2 cm on average
Cane color: RHA 176A
Leaves:
Terminal leaflet:
   Size.—Length: 10.5 cm (primocane) Width: 7.4 cm (primocane) Length/width ratio: 1.42.
   Shape of apex.—Acuminate.
   Shape of base.—Cordate.
   Margin.—Doubly-serrate.
   Texture.—Glabrous.
   Number of serrations/leaf.—128.
   Shape of serrations.—Acuminate.
   Color.—Upper surface: RHS N137A Lower surface: RHS N138C.
   Venation pattern.—Netted pinnate.
   Venation color.—Upper surface: RHS N137B Lower surface: RHS 145B.
   Shape of leaf in cross section.—Odd pinnate; trifoliate and pentafoliate.
   Number of leaflets/leaf.—3 to 5.
   Interveinal blistering.—No.
   Glossiness.—Yes.
Primocane leaves:
   Petiole length.—4.8 cm on average.
   Petiole diameter.—0.15 cm (primocane).
   Petiole color (upper).—RHS 177A (primocane).
   Petiole color (lower).—RHS 145B (primocane).
   Rachis length.—7.3 cm on average.
   4 Stipules/leaf.—2 on average, if present.
   Stipule length.—0.6 cm.
   Stipule width.—0.08 cm.
   Stipule color.—RHS 144A.
   Leaf color.—Leaf Upper surface: RHS N137A Leaf Lower surface: RHS N138C.
   Terminal leaflet.—Length: 10.5 cm on average Width: 7.4 cm on average Petiolule length: 2.7 cm on average Petiolule diameter: 0.09 cm Petiolule color (upper): RHS 177A Petiolule color (lower): RHS 145A.
   Distal lateral leaflet.—Length: 5.9 cm on average Width: 2.4 cm on average Petiolule length: 0 cm (attached to rachis).
   Basal lateral leaflet.—Length: 7.3 cm on average Width: 4.9 cm on average Petiolule length: 0 cm (attached to rachis).
Floricane leaves: (Floricane data recorded on Apr. 2, 2011)
   Petiole length.—3.4 cm on average.
   Stipule length.—0.5 cm, when present.
   Color.—Leaf Upper surface: RHS 144A Leaf Lower surface: RHS 148B.
   Terminal leaflet.—Length: 7.2 cm on average Width: 5.8 cm on average Petiolule length: 2.0 cm on average.
   Basal lateral leaflet.—Length: 6.8 cm on average Width: 3.3 cm on average Petiolule length: 0 cm (attached to rachis).
Flowers:
Time of flowering (50% of plants at first flower): 10-June (primocanes, year one); 6-April (floricanes, year two).
Size:
   Length.—0.9 cm on average.
   Diameter.—0.7 cm on average.
Fragrance: None
Color: (Please see R.H.S. values for petal, stamen color)
Peduncle:
   Length (cm).—1.3 cm on average.
   Diameter (cm).—0.7 cm on average.
   Color.—RHS 139C.
   Pubescence.—Present.
   Texture.—Pubescent.
Petal color: RHS 157A (both topside and underside)
Petals/flower: 5
Petal shape: Obtuse
Petal length: 0.9 cm
Petal width: 0.2 cm
Petal apex descriptor: Obtuse
Petal base descriptor: Obtuse
Petal margin descriptor: Entire, with some undulation near apex
Flowering trusses shape: Deltoid
Sepal:
   Quantity.—5.
   Length.—1.4 cm on average.
   Width.—0.3 cm on average.
   Color.—Topside: RHS 147C; Underside: RHS 139C.
   Sepal apex descriptor.—Acuminate.
   Sepal margin descriptor.—Entire.
Pedicel:
   Color.—RHS 146B.
   Length.—5.9 cm per flower on average.
   Pedicel diameter.—0.05 cm.
Receptacle color: RHS 159A
Reproductive organs:
Self fertile: Yes
Stamens:
   Number.—141 on average.
Filament:
   Length.—0.3 cm on average.
   Diameter.—0.06 cm on average.
Stamen color: RHS NN155B
Anthers:
   Color.—RHS 197A.
   Length.—0.09 cm on average.
   Diameter.—0.02 cm on average.
Pollen color: RHS 157D
Pollen amount: Sparse
Style:
   Length.—0.2 cm on average.
   Diameter.—0.01 cm on average.
   Color.—RHS 157B.
Stigma:
   Length.—0.01 cm on average.
   Diameter.—0.01 cm on average.
   Color.—RHS 156B.
Fruit:
Predominant shape: Conic
Weight (g): 5.1-5.4 g, on average
Size:
   Length (cm).—3.4 cm, on average.
   Width (cm).—1.9 cm, on average.
   Length/width ratio.—1.63.
Receptacle:
   Diameter.—1.0 cm, on average.
   Length.—2.0 cm, on average.
Drupelet:
   Diameter.—0.4 cm, on average.
   Width.—0.3 cm, on average.
   Number.—134 to 156.
   Weight.—0.02 g, on average.
Color of mature fruit:
   External.—RHS 42B.
   Internal.—RHS 48B.
Firmness of skin: Moderate
Firmness of flesh: Moderate in Watsonville, Calif., USA to Firm in Zapotiltic, Jalisco, Mexico
Hollow center: Yes
Number of fruit per node: 1 to 2
Time of ripening (50% of plants with first ripe fruit): 20-May (floricanes); 25-July (primocanes, year one)
Time of fruiting: Late spring, early summer (floricanes); Late summer, autumn (primocanes)
Type of bearing: Primocane-fruiting
Exemplary fruit yield: 16,307-18,200 lb/acre, on average
Berry brix: 8.8
Buds:
Bud shape: Acute
Bud length: 0.81 cm
Bud diameter (base): 0.5 cm
Bud diameter (tip): 0.1 cm
Bud color: RHS 144A
Typical market use: Fresh
Keeping quality: Very good
Shipping quality: Moderate to Good

What is claimed is:

1. A new and distinct cultivar of Raspberry plant named ‘Pacific Majesty’ as described and shown herein.
